SEQUENCE LISTING (43 sequences)

SEQ ID NO: 1 - DNA, 35 bp, synthetic construct
  source 1..35: mol_type = other DNA
ctagtctagaaccatgtgggtgcttacacctgctg

SEQ ID NO: 2 - DNA, 24 bp, synthetic construct
  source 1..24: mol_type = other DNA
atcatggaagacgccaaaaacata

SEQ ID NO: 3 - DNA, 20 bp, synthetic construct
  source 1..20: mol_type = other DNA
gtgcacggcgatctttccgc

SEQ ID NO: 4 - DNA, 20 bp, synthetic construct
  source 1..20: mol_type = other DNA
gtgcacggcgatctttccgc

SEQ ID NO: 5 - DNA, 27 bp, synthetic construct
  source 1..27: mol_type = other DNA
atctttgctgtattcaacttaacaatg

SEQ ID NO: 6 - DNA, 20 bp, synthetic construct
  source 1..20: mol_type = other DNA
atggtgagcaagggcgagga

SEQ ID NO: 7 - DNA, 24 bp, synthetic construct
  source 1..24: mol_type = other DNA
atcatggaagacgccaaaaacata

SEQ ID NO: 8 - DNA, 39 bp, synthetic construct
  source 1..39: mol_type = other DNA
ctagaccggttttgctgtattcaacttaacaatgaattg

SEQ ID NO: 9 - DNA, 40 bp, synthetic construct
  source 1..40: mol_type = other DNA
gagaccaagaaacgtgcagaattcgggtcggcatggcatc

SEQ ID NO: 10 - DNA, 19 bp, synthetic construct
  source 1..19: mol_type = other DNA
tgtaaaacgacggccagtg

SEQ ID NO: 11 - DNA, 23 bp, synthetic construct
  source 1..23: mol_type = other DNA
attctgcacgtttcttggtctct

SEQ ID NO: 12 - DNA, 22 bp, synthetic construct
  source 1..22: mol_type = other DNA
gctctattagtcaccggatggc

SEQ ID NO: 13 - DNA, 23 bp, synthetic construct
  source 1..23: mol_type = other DNA
gtgcttctgctaggatcaatgtg

SEQ ID NO: 14 - DNA, 20 bp, synthetic construct
  source 1..20: mol_type = other DNA
cgacctgatctacactgggg

SEQ ID NO: 15 - DNA, 22 bp, synthetic construct
  source 1..22: mol_type = other DNA
tacctttgtgcgcctgttttat

SEQ ID NO: 16 - DNA, 20 bp, synthetic construct
  source 1..20: mol_type = other DNA
cgacctgatctacactgggg

SEQ ID NO: 17 - DNA, 20 bp, synthetic construct
  source 1..20: mol_type = other DNA
taagggagagcaagcttggc

SEQ ID NO: 18 - DNA, 20 bp, synthetic construct
  source 1..20: mol_type = other DNA
attccctctcgttcgaaccg

SEQ ID NO: 19 - DNA, 20 bp, synthetic construct
  source 1..20: mol_type = other DNA
gagaccgcaactgaaaagtt

SEQ ID NO: 20 - DNA, 20 bp, synthetic construct
  source 1..20: mol_type = other DNA
ctctggcgttgacttttcaa

SEQ ID NO: 21 - DNA, 16 bp, synthetic construct
  source 1..16: mol_type = other DNA
aagaaacgtgcagaat

SEQ ID NO: 22 - DNA, 16 bp, synthetic construct
  source 1..16: mol_type = other DNA
ttctttgcacgtctta

SEQ ID NO: 23 - DNA, 20 bp, synthetic construct
  source 1..20: mol_type = other DNA
gagaccgcaactgaaaagtt

SEQ ID NO: 24 - DNA, 20 bp, synthetic construct
  source 1..20: mol_type = other DNA
ctctggcgttgacttttcaa

SEQ ID NO: 25 - RNA, 16 nt, synthetic construct
  source 1..16: mol_type = other RNA
taagggagagcaagct

SEQ ID NO: 26 - RNA, 16 nt, synthetic construct
  source 1..16: mol_type = other RNA
attccctctcgttcga

SEQ ID NO: 27 - RNA, 20 nt, synthetic construct
  source 1..20: mol_type = other RNA
gagaccgcaactgaaaagtt

SEQ ID NO: 28 - RNA, 20 nt, synthetic construct
  source 1..20: mol_type = other RNA
ctctggcgttgacttttcaa

SEQ ID NO: 29 - RNA, 16 nt, synthetic construct
  source 1..16: mol_type = other RNA
aagaaacgtgcagaat

SEQ ID NO: 30 - RNA, 16 nt, synthetic construct
  source 1..16: mol_type = other RNA
ttctttgcacgtctta

SEQ ID NO: 31 - RNA, 20 nt, synthetic construct
  source 1..20: mol_type = other RNA
gagaccgcaactgaaaagtt

SEQ ID NO: 32 - RNA, 20 nt, synthetic construct
  source 1..20: mol_type = other RNA
ctctggcgttgacttttcaa

SEQ ID NO: 33 - RNA, 36 nt, synthetic construct
  source 1..36: mol_type = other RNA
taagggagagcaagctgagaccgcaactgaaaagtt

SEQ ID NO: 34 - RNA, 36 nt, synthetic construct
  source 1..36: mol_type = other RNA
aagaaacgtgcagaatgagaccgcaactgaaaagtt

SEQ ID NO: 35 - RNA, 20 nt, synthetic construct
  source 1..20: mol_type = other RNA
agaaacgtgcagaattcggg

SEQ ID NO: 36 - RNA, 20 nt, synthetic construct
  source 1..20: mol_type = other RNA
tctttgcacgtcttaagccc

SEQ ID NO: 37 - RNA, 20 nt, synthetic construct
  source 1..20: mol_type = other RNA
gagaccgcaactgaaaagtt

SEQ ID NO: 38 - RNA, 20 nt, synthetic construct
  source 1..20: mol_type = other RNA
ctctggcgttgacttttcaa

SEQ ID NO: 39 - RNA, 16 nt, synthetic construct
  source 1..16: mol_type = other RNA
agaaacgtgcagaatt

SEQ ID NO: 40 - RNA, 16 nt, synthetic construct
  source 1..16: mol_type = other RNA
tctttgcacgtcttaa

SEQ ID NO: 41 - RNA, 20 nt, synthetic construct
  source 1..20: mol_type = other RNA
gagaccgcaactgaaaagtt

SEQ ID NO: 42 - RNA, 20 nt, synthetic construct
  source 1..20: mol_type = other RNA
ctctggcgttgacttttcaa

SEQ ID NO: 43 - RNA, 36 nt, synthetic construct
  source 1..36: mol_type = other RNA
agaaacgtgcagaattgagaccgcaactgaaaagtt

(19) United States
(12) Patent Application Publication
(10) Pub. No.: US 2026/0234686 A1
(43) Pub. Date: Aug. 13, 2026

(54) CIRCULAR RNA PRODUCTION METHODS, RNA MOLECULES, AND USES THEREOF

(71) Applicant: National Health Research Institutes, Miaoli County (TW)

(72) Inventor: Chia-Yi Yu, Miaoli County (TW)

(21) Appl. No.: 19/498,332

(22) PCT Filed: Nov. 8, 2023
(86) PCT No.: PCT/US2023/079115

§ 371 Date: Dec. 30, 2025

Related U.S. Application Data
(60) Provisional application No. 63/423,542, filed on Nov. 8, 2022.

(51) Int. Cl. C12P 19/34; C12N 15/113

(52) U.S. Cl. CPC C12P 19/34; C12N 15/113

(57) ABSTRACT

A method for covalently joining linear RNA molecules end-to-end to extend their functionality. A cis-acting ligase ribozyme is used to generate circular RNA in vitro for ectopic protein expression. RNA circularization was confirmed by masking the 5′ phosphate group, resisting exonuclease RNase R digestion, and sequencing the RT-PCR products of the joined region. Furthermore, one internal ribosome entry site rendered oRNA translation competent, but two IRES in cis, not tans, hampered the translation and an oRNA expressing ribonuclease Cas13 showed excellent potential against the corresponding RNA virus. A method for using oRNA is also provided.

### BACKGROUND OF THE INVENTION

### Technical Field of the Invention

The present invention relates to circular RNA molecules, vectors, method of producing circular RNA, and utilization methods.

### Background

The success of introducing messenger RNA (mRNA) encoding SARS-CoV-2 spike protein as a novel vaccine strategy undoubtedly sparks the potential of RNA research in molecular therapy. In theory, the longer the half-life of delivered mRNA within the cell, the extended duration of its functionality, and the reduced amount of mRNA needed for delivery. Most mRNAs have 5′-Cap and 3′ poly(A) in conjunction with various cellular proteins to reach each other and form a loop structure for efficient protein expression and avoiding exonuclease digestion. In response to elevating translation efficiency and longevity of the exogenous mRNA in cells, covalently circularizing single-stranded mRNA to generate the circular RNA (aka circRNA or oRNA) became a new choice.

Production of circular RNA (aka circRNA or oRNA) molecules has been attempted by various methods. However, the process is intricate and necessitates extensive purification. One popular oRNA formation strategy, the permuted intron-exon (PIE) splicing, involves fused partial exons flanked by intron and corresponding complementary sequences. When the RNA elements of the intron, splice site, and exon are permuted correctly, the RNA acts as an enzyme, the so-called ribozyme, catalyzes the splicing event to form oRNA via RNA breakdown and re-ligation. The PIE splicing strategy tolerates linear RNA in large and can be performed in vitro and in vivo but consumes quota in RNA sequence length for being the spliced wastes. Despite a particular condition with GTP and Mg²⁺ accelerating PIE splicing in vitro, small byproduct RNAs after splicing required additional removal procedures to eliminate potential side effects.

Therefore, the present invention provides a method of oRNA synthesis by a cis-acting ligase ribozyme (Rz^{L}). Unlike the splicing method of acquiring RNA breakdown and re-ligation, the present invention depends on ligase ribozyme RNA structure to ligate RNA directly. That said, neither RNA breakdown is needed, nor byproduct RNA is generated. Also, the present invention further provides a circular RNA and uses thereof.

### SUMMARY OF INVENTION

One aspect of the present invention provides one or more plasmids having an arrangement of the region, wherein said arrangement is a 5′ polymerase promoter sequence, a nucleic acid sequence encoding a ligase ribozyme RNA, a nucleic acid sequence encoding an internal ribosome entry site (IRES), a nucleic acid sequence encoding an effector molecule, and a nucleic acid harboring a free OH group at the very 3′ end for conjugation to the ligase ribozyme RNA aforementioned.

Another aspect of the present invention relates to a method of producing a circular RNA molecule comprising the steps of: (a) providing a plasmid; (b) transcribing an RNA base on the plasmid and (c) allowing the RNA to self-ligation, thereby producing the circular RNA; wherein the plasmid comprises a 5′ polymerase promoter sequence, a ligase ribozyme RNA sequence, an IRES sequence, a nucleic acid sequence encoding an effector molecule, and a nucleic acid harboring a free OH group at the very 3′ end for conjugation to the ligase ribozyme RNA aforementioned.

In some embodiments of the present invention, the ligase ribozyme RNA sequence is a cis-acting ligase ribozyme sequence.

In some embodiments of the present invention, the 5′ polymerase promoter sequence is prokaryotic T7 promoter, T3 promoter, SP6 promoter, Lac promoter, or eukaryotic RNA polymerase I promoter.

In some embodiments of the present invention, the IRES sequence is selected from a virus, a cell, an artificial sequence, or a gene selected from the group consisting of CVB3 virus, EV71 virus, EMCV virus, PV virus, and CSFV virus.

In some embodiments of the present invention, the effector molecule sequence is microRNA sequence, microRNA sponge sequence, receptor protein sequence, target protein sequence, antigen sequence, protein enzyme sequence, functional protein sequence, or bicistronic protein expression sequence.

In some embodiments of the present invention, the arrangement further comprises a substrate sequence of the ligase ribozyme RNA.

In some embodiments of the present invention, the substrate sequence of the ligase ribozyme is substrate sequence, substrate_2 sequence, or substrate_3 sequence.

A further aspect of the present invention is a circular RNA produced by the present method.

Still yet another aspect of the present invention relates to a protein expression method. This method involves using one or more plasmids from the present invention under conditions effective to express the effector molecules.

Described infra is a novel system for generating circular RNAs in virtually any metazoan or bacterial cell. In one embodiment, this can be done without the co-expression of any additional proteins or enzyme. This expression system takes advantage of the ligase ribozyme with related functions, which autonomously process the ligation of RNA 5′ and 3′ end.

### BRIEF DESCRIPTION OF THE DRAWINGS

FIG. 1A illustrates a DNA template with elements for the PIE strategy carrying out the group I intron self-splicing as the conventional oRNA production method. Note the XbaI and StuI restriction enzyme recognition sites were applied in the DNA template linearization before RNA synthesis. IVT, in vitro transcription; T7, T7 promoter; homo, homology arm; IRES, internal ribosome entry site of coxsackievirus B3; GOI, gene of interest.

FIG. 1B illustrates the precursor linear RNA produced by IVT, and the subsequent RNA circularization through PIE splicing generates target oRNA, which is resistant to digestion by RNase R but susceptible to RNase A.

FIG. 1C-1D depict the analysis of IVT products from either XbaI- or StuI-linearized DNA templates, following treatment with the specified enzymes. FIG. 1C shows the results of agarose electrophoresis. FIG. 1D shows the quantification results of band intensity, which were measured using ImageJ.

FIG. 1E provides a schematic illustration of the RT-PCR primer sets used for detecting the target oRNA. Arrows indicate the primer orientation from 5′-end to 3′-end with specific regions of the oRNA targeted.

FIG. 1F illustrates that IVT products from the XbaI- or StuI-linearized template were purified, circularized with indicated conditions, digested with RNase R, and then sampled for RT-PCR measuring oRNA. The PCR products were analyzed by agarose electrophoresis.

FIG. 1G shows the quantitative PCR (qPCR) result using SYBR Green dye (data are mean±SD, n=3 per group, and were compared by two-tailed Student t test).

FIG. 1H-J show that IVT products from the StuI-linearized template were incubated in the indicated condition (GTP, 2 mM; 55° C., 15 min) and subsequently analyzed. FIG. 1H presents the analysis results of agarose electrophoresis. FIG. 1I presents the analysis results of RT-PCR. FIG. 1J shows the quantification results of RT-qPCR (data are mean #SD, n=3 per group, and were compared by two-tailed Student t test).

FIG. 2A-2E exhibit the cis-acting ligase ribozyme (Rz^{L}) system in this invention. FIG. 2A is a schematic illustration of a pair of Rz^{L} and substrate sequence (SUB) flanking the IRES-driven protein expression cassette. The RNA product could undergo circularization autonomously while in the IVT reaction. FIG. 2B exhibits the design of DNA template with different ends for IVT. FIG. 2C-2E show the analysis of IVT products from different DNA templates. FIG. 2C shows the results of agarose electrophoresis. FIG. 2D shows the quantification results of RT-qPCR (data are mean±SD, n=3 per group, and were compared by two-tailed Student t test). FIG. 2E shows the sequencing data as indicated.

FIG. 2F shows the RNA folding prediction of the Rz^{L} and the designed endings.

FIG. 3A-3B illustrate the impact of N7-methyl-guanosine-5′-ppp-5′-guanosine (5′-Cap) addition (0%, 80%, and 90%) in the IVT on the analyzed RNA products. FIG. 3A depicts the analysis of IVT products via agarose electrophoresis. FIG. 3B shows the quantification results of agarose electrophoresis. (Data are mean±SD (n=3 per group) and were compared by two-tailed Student t test).

FIG. 3C provides a schematic illustration of the DNA template that ended with SUB, which was either transfected into the BHK-21 cells stably expressing T7 RNA polymerase (BHK-T7) or subjected for IVT followed by RNA transfection into the parental BHK-21 cells.

FIG. 3D shows the analysis of oRNA using RT-PCR.

FIG. 3E shows the images of transfected cells using fluorescence microscopy.

FIG. 3F exhibits the intensity of RFP-encoded oRNA measured by flow cytometry.

FIG. 4A-4F show that RNA modification could reduce the Rz^{L}-catalyzed oRNA formation. FIG. 4A-4B show that the Rz^{L}-SUB pair was highlighted with dark circles to indicate possible N6-methyladenosine (m6A) (FIG. 4A) or 5-methylcytosine (m5C) (FIG. 4B) modifications. Note that the content of A in the pair is higher than C. FIG. 4C-4F shows that different ratios of m6A- or m5C-modified A or C were added in the IVT whose RNA products were analyzed for its RNase R resistance (FIG. 4C) and oRNA formation (FIG. 4C-FIG. 4D) as indicated. Data are mean±SD (n=3 per group). FIG. 4E-4F show that BHK-21 cells were transfected by the IVT products with the indicated ratio of m6A- or m5C-modified RNA, followed by analysis using fluorescence microscopy (FIG. 4E) and flow cytometry (FIG. 4F).

FIG. 5A-5E show that an extraneous IRES in cis, not trans, hampered the oRNA translation. FIG. 5A illustrates the strategy for designing mono- and bicistronic oRNA constructs to express the indicated reporter proteins. FIG. 5B shows that N18 cells were transfected with the indicated oRNA for 24 h and harvested for FLuc activity (data are mean±SD, n=3 per group). FIG. 5C illustrates the analysis of reporter protein expression using western blotting. FIG. 5D illustrates the analysis of reporter protein expression using flow cytometry. FIG. 5E illustrates the analysis of reporter protein expression using fluorescence microscopy.

FIG. 6A-6D show that an IRES-driven oRNA expressing erCas13 could be antiviral against the recombinant RNA virus JEV-GFP. FIG. 6A shows the use of fluorescence microscopy to observe N18 cells infected with JEV-GFP virus (moi 10) or transfected with oRNA (1 ug) for the indicated time. FIG. 6B is a schematic illustration of timepoint of JEV-GFP virus (moi 10) infection and oRNA transfection. FIG. 6C-6D depict the results of flow cytometry analysis for JEV-GFP virus (moi 10) infection and oRNA transfection.

FIG. 6E shows that N18 cells were co-transfected with DNA plasmid encoding erGFP and oRNA encoding Flag-tagged Cas13 as indicated.

FIG. 6F-6H shows that N18 cells were treated with oRNA, JEV-GFP, or oRNA at the time and with the dose as indicated (FIG. 6F), which was analyzed by Western blotting (FIG. 6G) and flow cytometry (FIG. 6H). The design of ER-targeting Cas13 in the oRNA was illustrated (FIG. 6G, lower panel). +, sgRNA targeting GFP; −, N18 cellular RNA.

FIG. 7A-7B show that Reticulocyte lysate was incubated with control FLuc mRNA (5′-Capped, 3′-polyA-tailed) for translation in vitro and then with CHX to cease. FIG. 7A shows that control conditions for CHX working concentrations were illustrated (upper panel) and tested (lower panel) as indicated (one of the represented results). FIG. 7B shows that time intervals were illustrated (upper panel) and tested (lower panel) as indicated (one of the represented results).

FIG. 7C-7D show that IVT products from either XbaI- or StuI-linearized pOR1 DNA templates were added to the reticulocyte lysate (data are mean±SD, n=2 per group) or transfected to BHK-21 cells (data are mean±SD, n=3 per group) for the indicated time.

FIG. 8A-8B demonstrate that based on the designed substrate (SUB) sequence, the Rz^{L}-SUB pair ends with an extra nucleotide could probably make overhanging at the 3′ end, except for additional U.

FIG. 8C shows the DNA template ended with either SUB or SUB^{EcoRI} for IVT.

FIG. 8D shows the sequencing result of IVT products from each DNA template.

FIG. 8E shows the agarose electrophoresis and RT-PCR result of oRNA (data are mean±SD, n=3 per group).

FIG. 8F shows the quantification results of RT-qPCR of oRNA (data are mean±SD, n=3 per group).

FIG. 8G depicts the RNA folding prediction of oRNA transcribed from the DNA template designed with SUB_2 sequence and SUB_3 sequence at the ends.

FIG. 8H shows the RT-PCR result of oRNA transcribed from the DNA template designed with SUB_2 sequence and SUB_3 sequence at the ends.

FIG. 9A is the schematic illustration of the experimental design. N18 cells were infected with JEV-GFP, transfected with oRNA harboring Vip or FLuc, and analyzed by flow cytometry.

FIG. 9B shows the JEV-GFP infection rate analyzed via flow cytometry.

FIG. 10A shows that N18 cells were co-transfected for 24 h with plasmid DNA encoding erGFP and oRNA expressing either Cas13 or erCas13, followed by immunofluorescence microscopy.

FIG. 10B-10C show that cells were treated with oRNA, JEV-GFP, or oRNA at the time and with the dose indicated (FIG. 10B), which was analyzed by Western blotting with indicated antibodies (FIG. 10C). +, sgRNA targeting GFP; −, N18 cellular RNA.

### DETAILED DESCRIPTION OF THE INVENTION

Other features and advantages of the present invention will be further exemplified and described in the following examples, which are intended to be illustrative only and not to limit the scope of the invention.

The embodiments of the invention will be apparent from the following detailed description, which proceeds with reference to the accompanying drawings, wherein the same references relate to the same elements.

As used herein, the term “promoter sequence” refers to T7 promoter, T3 promoter, SP6 promoter, CMV promoter, SV40 promoter, Lac promoter, eukaryotic RNA polymerase I promoter.

As used herein, the term “ligase ribozyme” refers to Ligase R1, Ribozyme L2, Ligate Z, Ribozyme X1, Ligase RibA, Circularize R, RibozymeLinker.

As used herein, the term “substrate sequence” refers to F1 substrate A sequence, F1 substrate 2 sequence, F1 substrate 3 sequence, F1 substrate B sequence, F1 substrate X sequence, F1 substrate Z sequence.

As used herein, the term “IRES sequence” refers to CVB3 virus sequence, EV71 virus sequence, EMCA virus sequence, PV virus sequence, CSFV virus sequence, cellular RNA sequence, and artificial RNA sequence.

### Example 1. The PIE Splicing Strategy Forms RNase R-Resistant oRNA

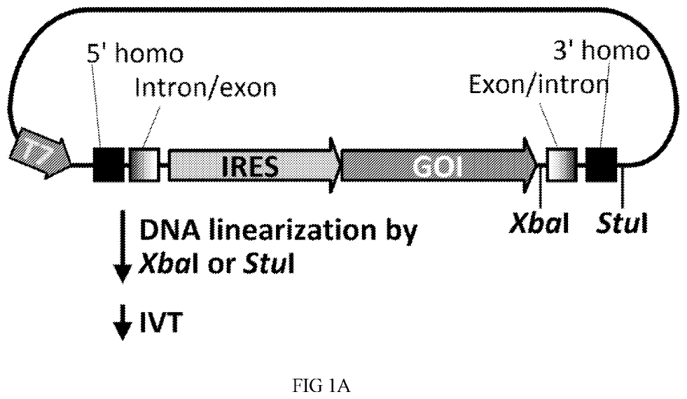

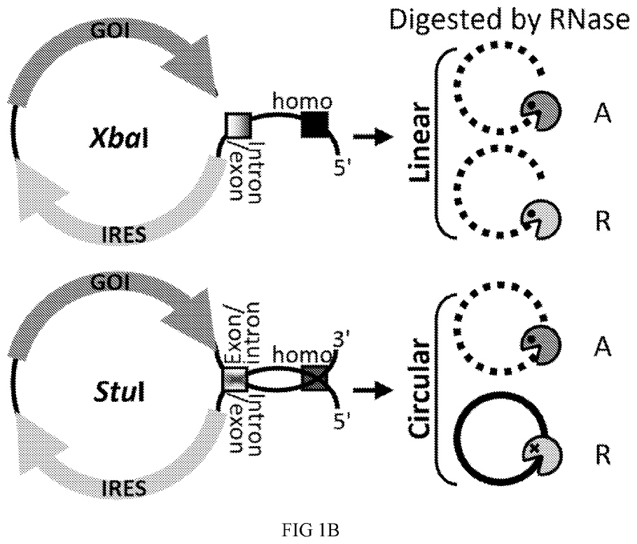

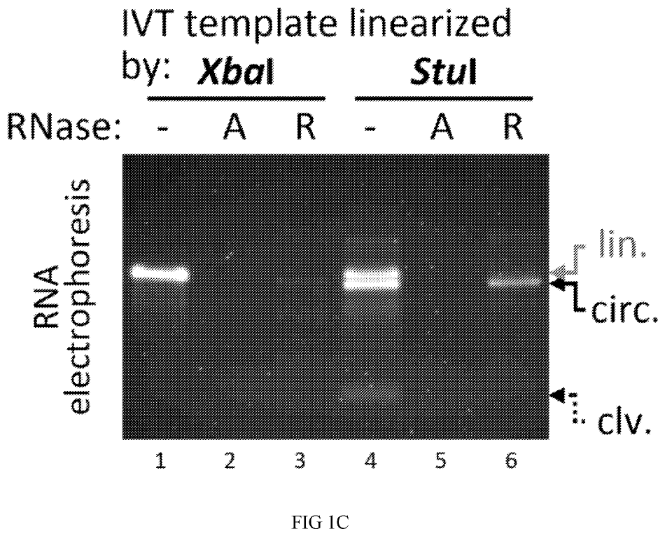

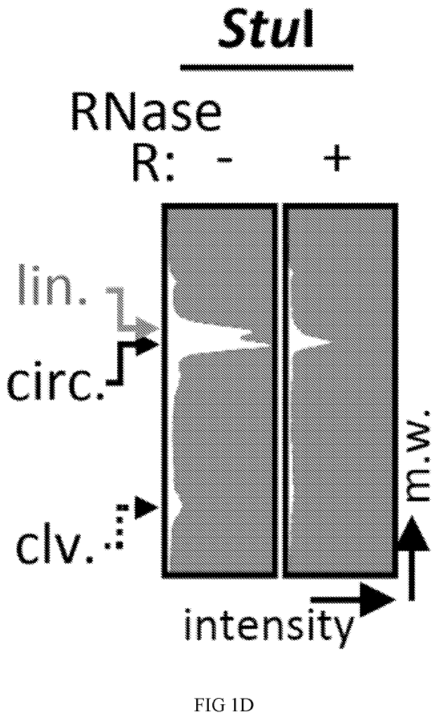

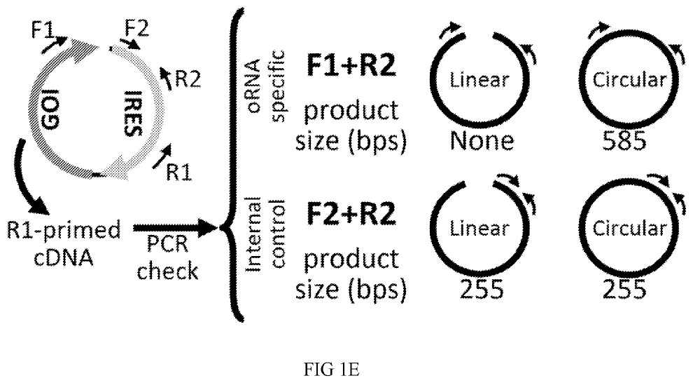

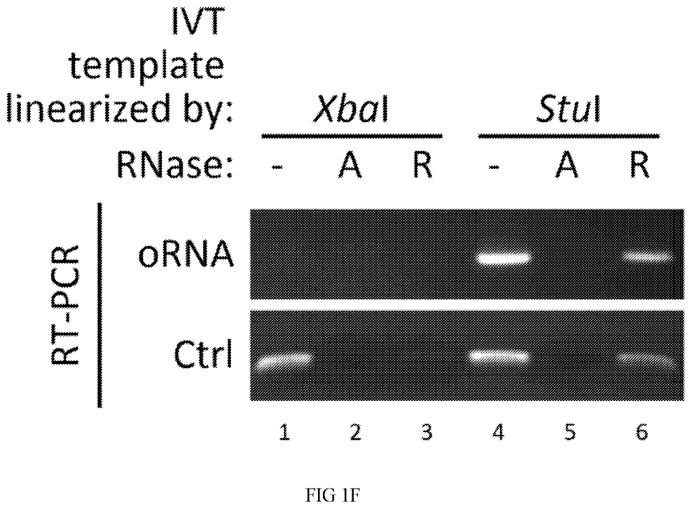

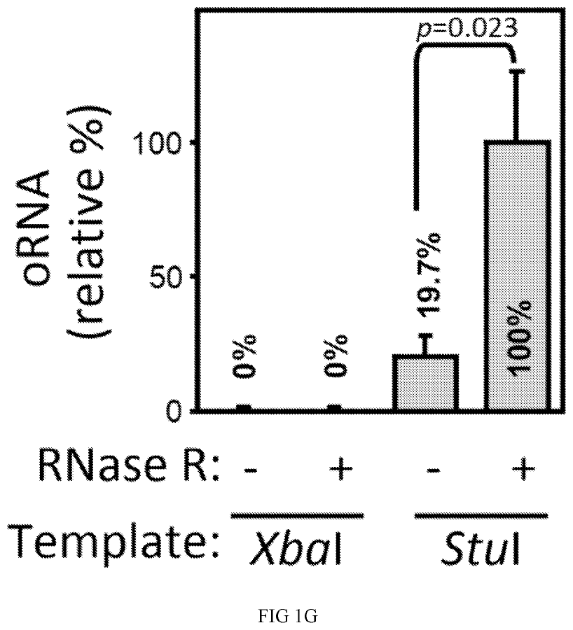

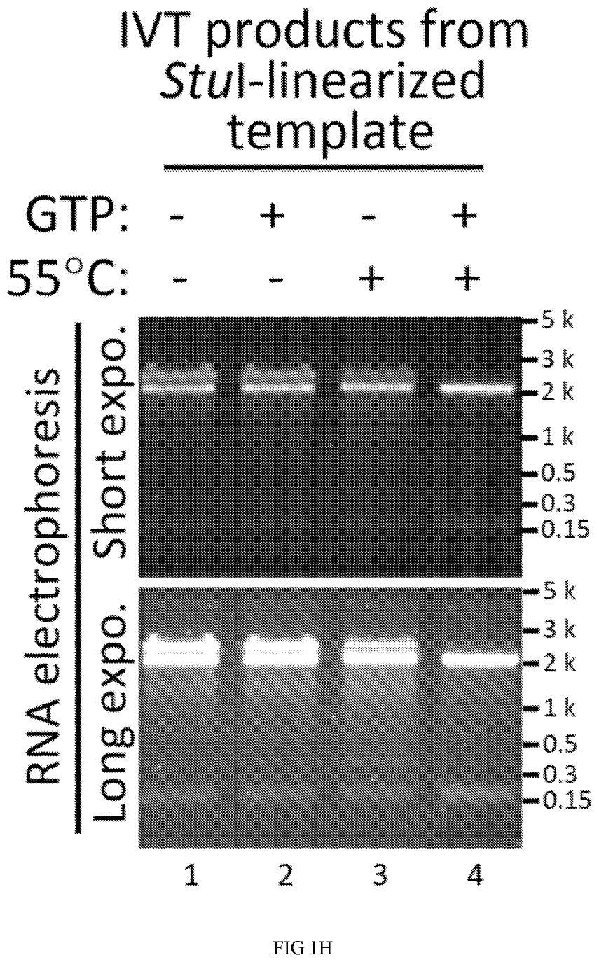

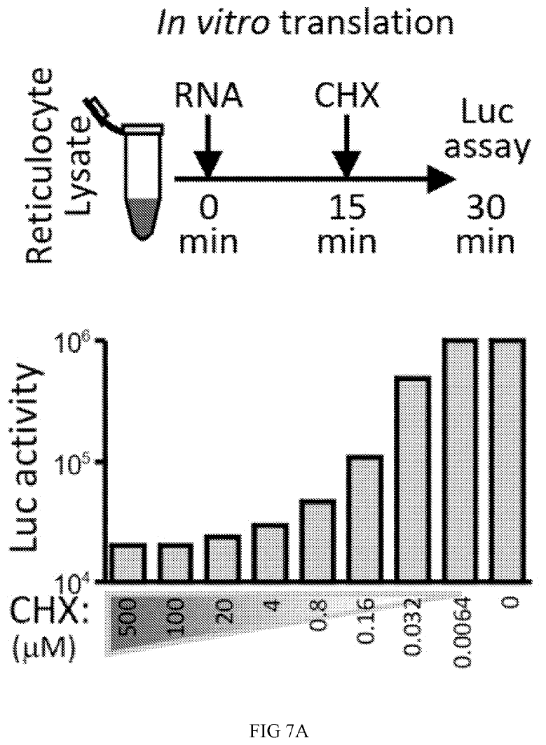

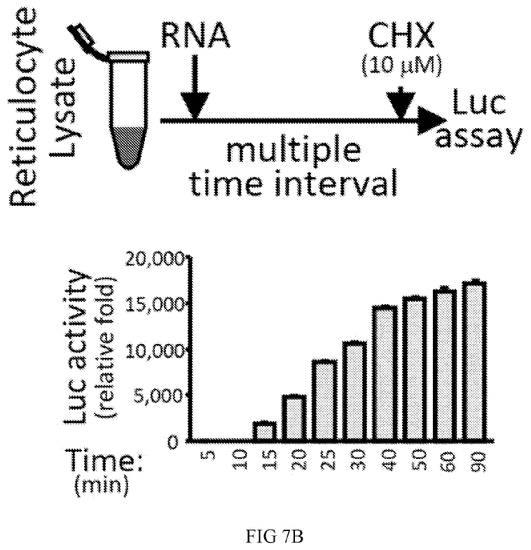

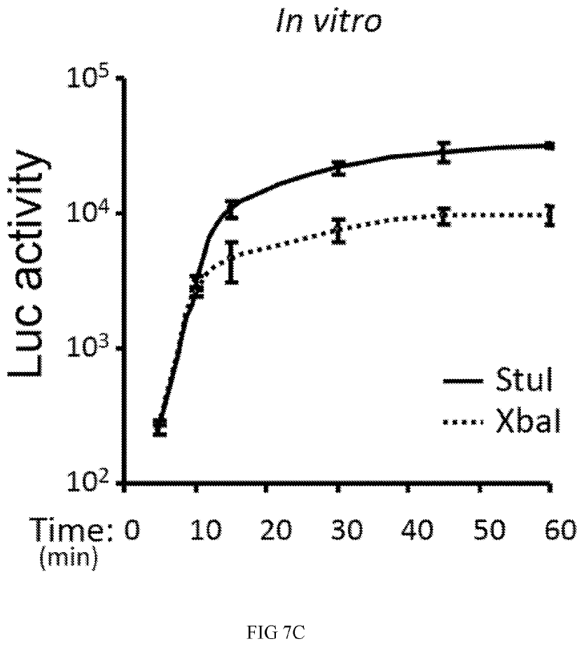

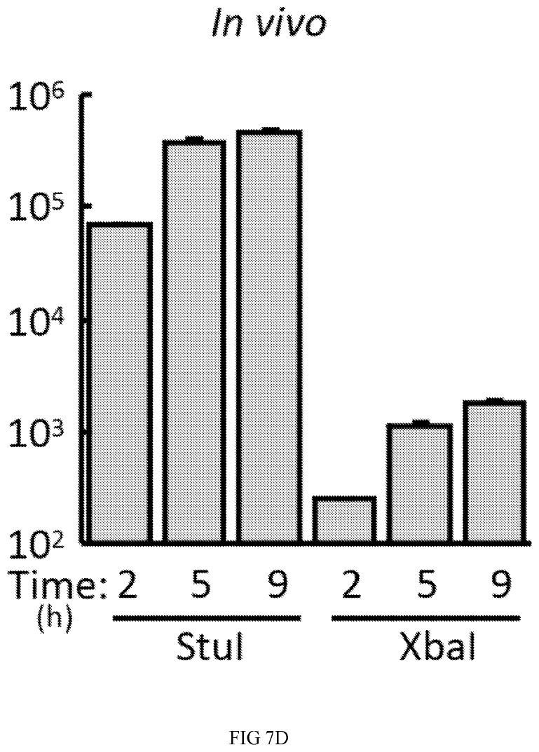

In this and following examples, the synthesized pOR1 vector comprises an IRES-driven protein expression cassette flanked by a sequence set of Anabaena catalytic intron/exon PIE system resided in a pair of homology arms under the control of T7 promoter (FIG. 1A). The restriction enzyme StuI-linearized template contains all the elements forming oRNA, whereas the XbaI-linearized template generates RNA lacking pairing elements and staying in linear form (FIG. 1B, left). RNase R, an exoribonuclease digesting linear RNA was applied to tell the oRNA formation because the RNA electrophoresis showed the XbaI- and StuI-linearized templates generating a variety of RNA species (FIG. 1C). Because of the difficulty of oRNA quantification using photo intensity (FIG. 1D), an RT-PCR method was designed with specific primers to measure oRNA (FIG. 1E). The RT-PCR consistently showed the StuI but not XbaI group of RNA forms oRNA as expected (FIG. 1F) despite low efficiency (FIG. 1G). RNA was heated in the presence of GTP for a better splicing activity generating oRNA (FIG. 1H-J). As expected, RNA from StuI-linearized templates expressed more reporter proteins in vitro and the transfected cells (in vivo) than that from XbaI-linearized ones (FIG. 7). Thus, oRNA production, qualification, and quantification by the conventional PIE splicing method were established.

### Example 2. A Ligase Ribozyme (Rz^{L}) Resided at RNA 5′-End Autonomously Catalyzes RNA Circularization in Virto

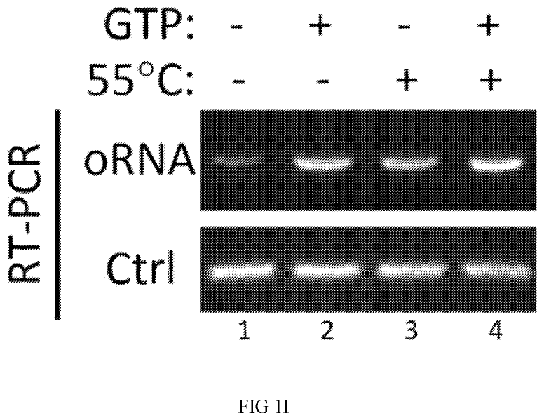

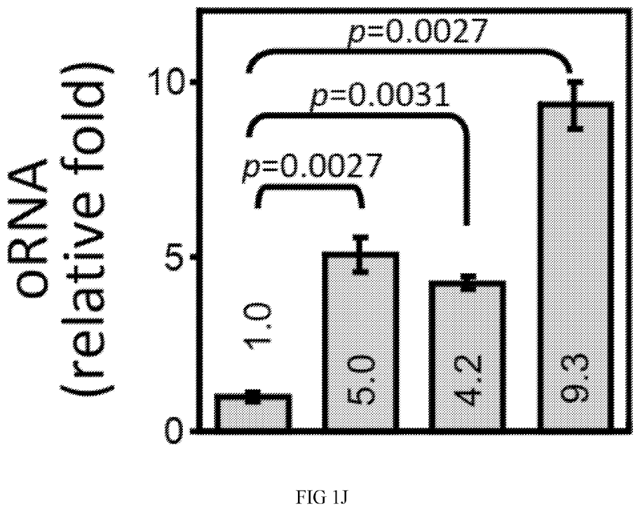

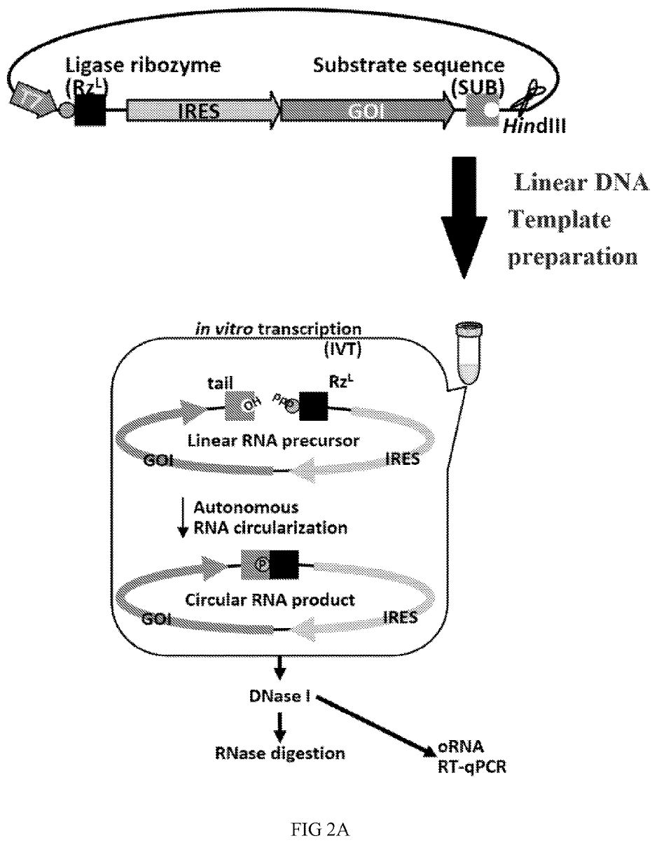

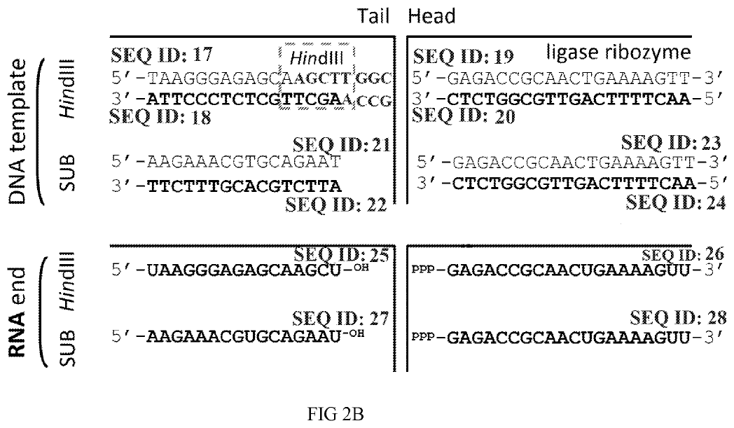

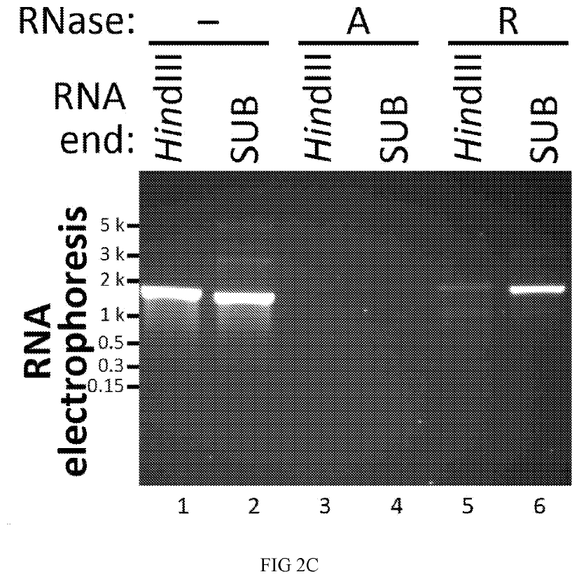

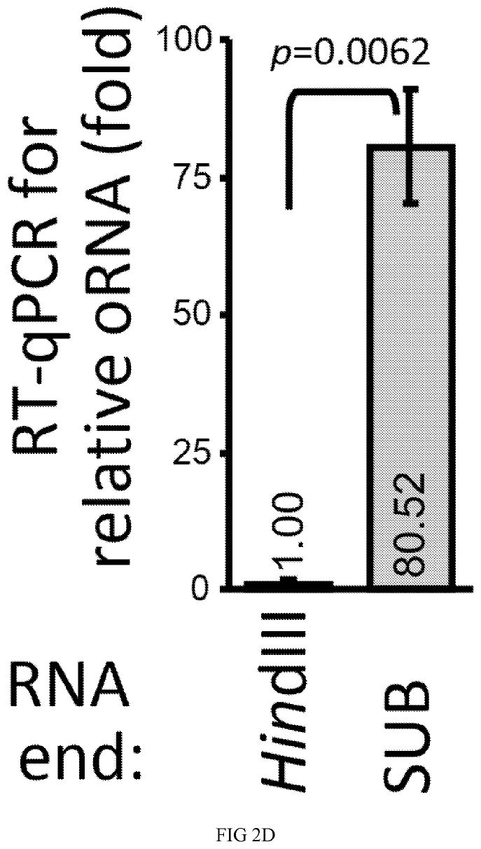

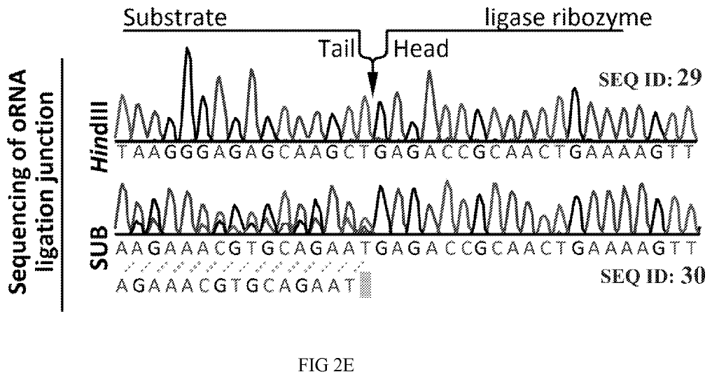

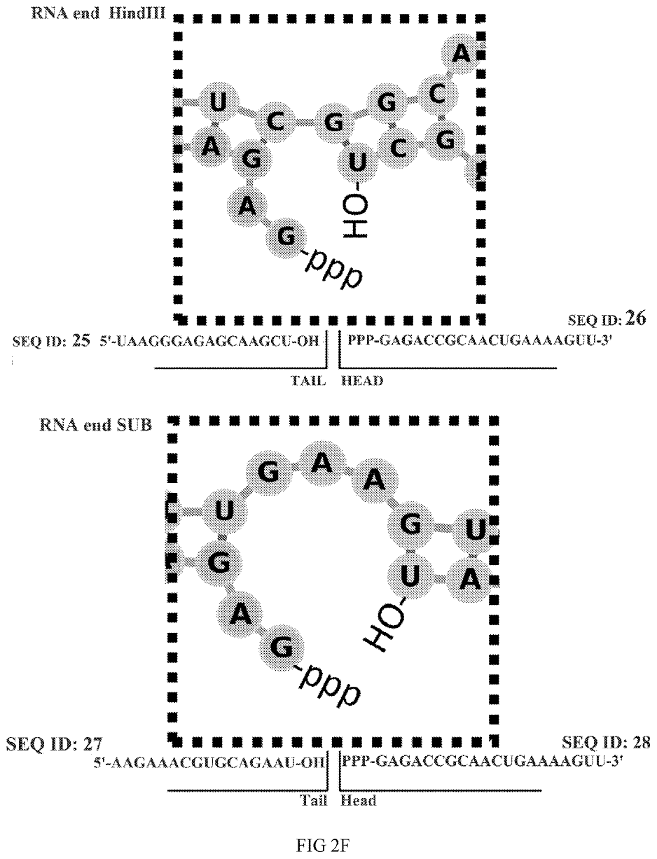

To generate oRNA without the splicing byproducts, the synthesized pOR2 vector contains the same protein translation cassette but is flanked by a pair of Rz^{L} and F1 substrate sequences (FIG. 2A). The HindIII linearization leaves the DNA template 5′-protruding ended with an imperfect but acceptable substrate sequence. For ending with the corresponding substrate RNA (SUB) fitting the Rz^{L}, PCR was used to generate a blunted-ended template that should end as designed (FIG. 2B). Both HindIII- and SUB-end templates seemed to generate equivalent RNA products (FIG. 2C, lanes 1 and 2) sensitive to RNase A digestion (FIG. 2C, lanes 3 and 4). However, the SUB-end group RNA was more RNase R-resistant than the HindIII group (FIG. 2C, lanes 5 and 6). oRNA-specific RT-qPCR consistently showed that the SUB-end group automatically formed more oRNA than the HindIII group in vitro transcription (IVT) (FIG. 2D). The PCR products were sequenced (FIG. 2E) to confirm details of the RNA covalent circularization design (FIG. 2F). Thus, RNA ending with the corresponding SUB sequence fitting the Rz-yielded oRNA efficiently, despite a minor presence of an extraneous nucleotide (FIG. 2E, star marked) that probably resulted from the none-5′ end-protruding IVT template.

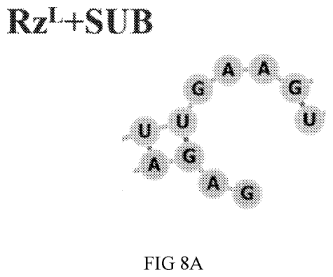

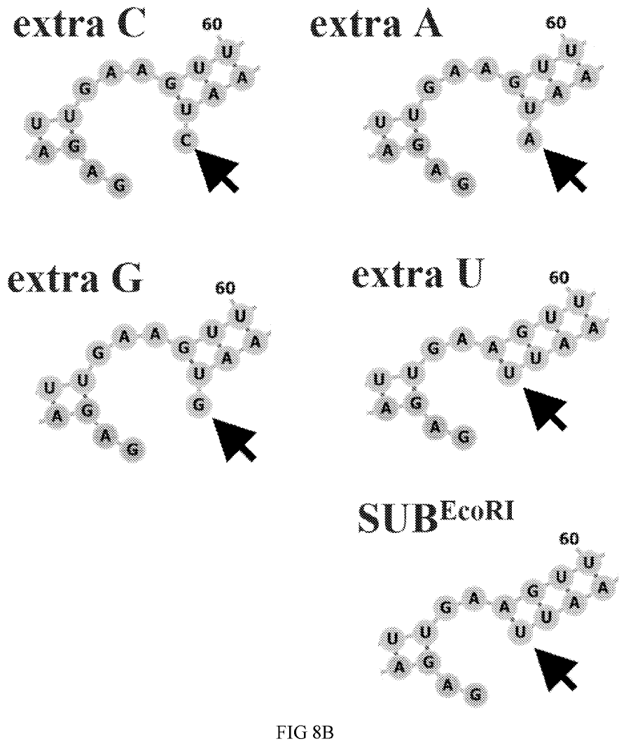

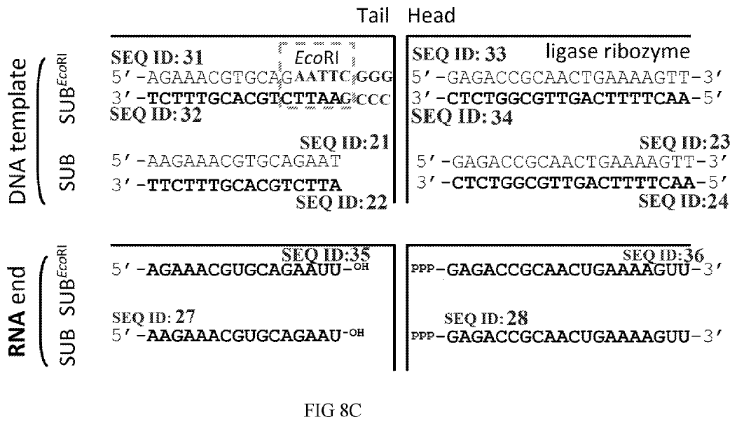

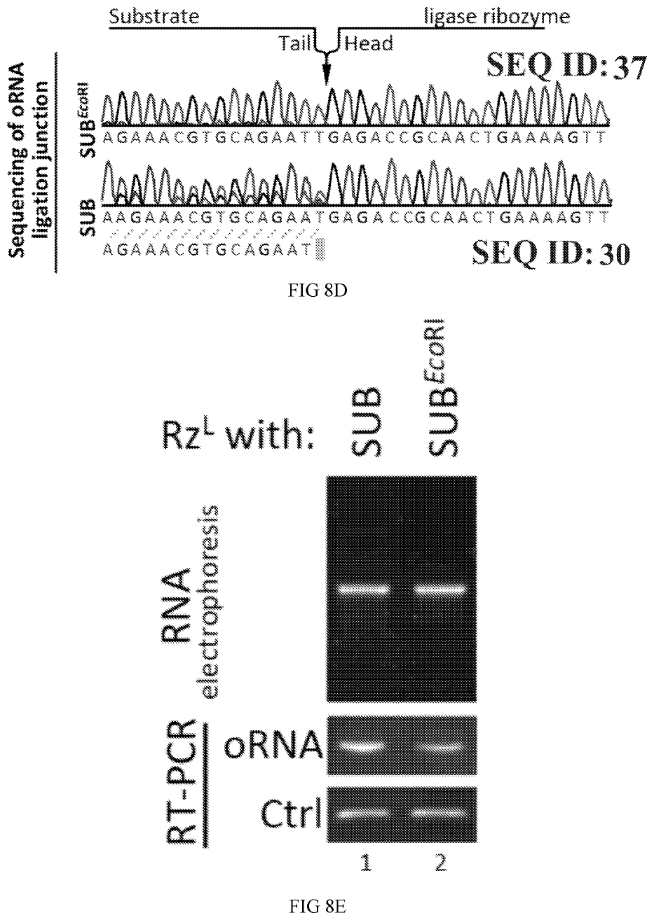

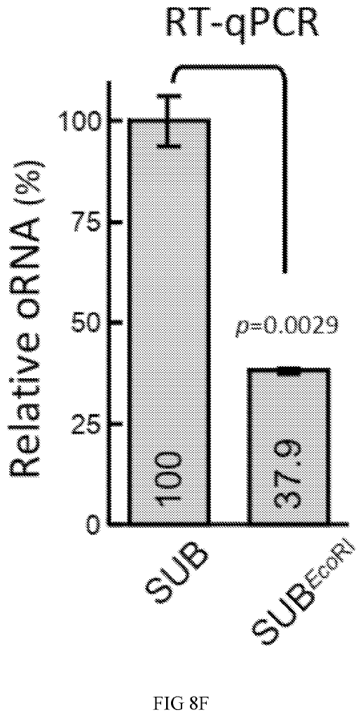

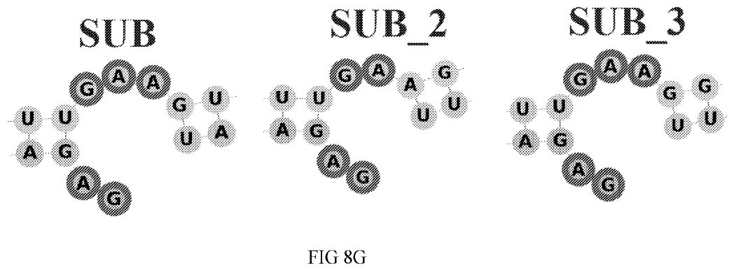

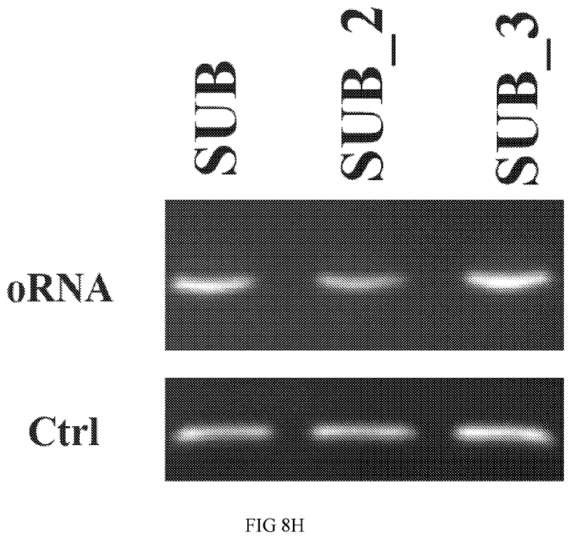

Since T is relatively higher than others in the extraneous nucleotide site (FIG. 2E, star), the RNA folding of each Rz^{L}-SUB pair which ends with a different extraneous nucleotide were analyzed (FIG. 8A, FIG. 8B). Because the extra U following SUB made the RNA end without an overhang that differs from the other three, the SUB template was ended with an EcoRI cutting site (SUB^{EcoRI}) to create a 5′ protruding end of AATT (FIG. 8C) for mimicking the circumstances with extra U (FIG. 8B). The SUB^{EcoRI} showed a precise ligation as expected (FIG. 8D) but with lower efficiency than SUB in RNA circularization (FIG. 8E, F). Thus, certain RNA substrate sequence tolerances existed beyond perfect matches in the Rz^{L}-SUB paired RNA ligation.

Optionally, the RNA molecules can have an ending that corresponds to the SUB sequence, SUB_2 sequence and SUB_3 sequence. In this optional configuration, the SUB sequence, the SUB_2 sequence and SUB_3 sequence are designed to form a conjunction ring with a 5′-end-protruding overhang end (FIG. 8G). The conjunction ring consists of three nucleotides for bridging at the middle and two overhang nucleotides at the 5′ end. This specific design has been found to efficiently yield oRNA (FIG. 8H).

### Example 3. RNA Modifications Affect the Rz^{L}-SUB oRNA Formation

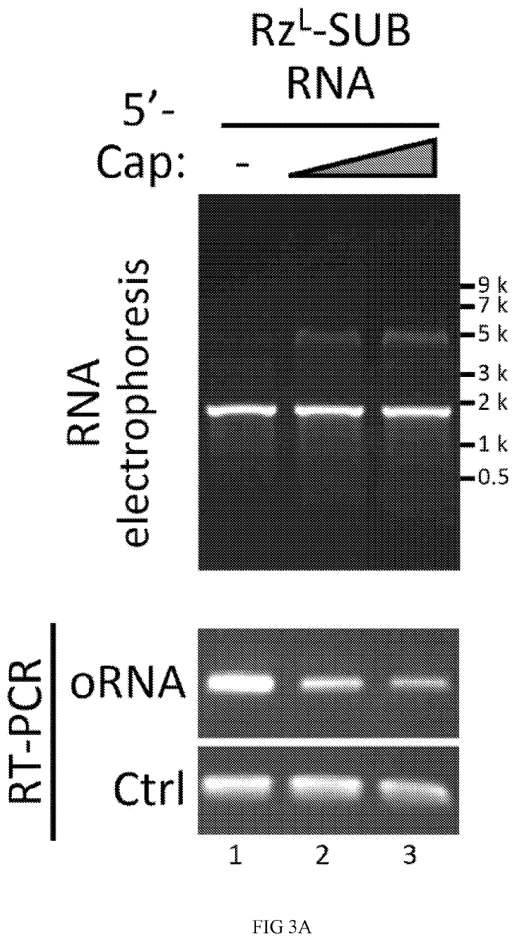

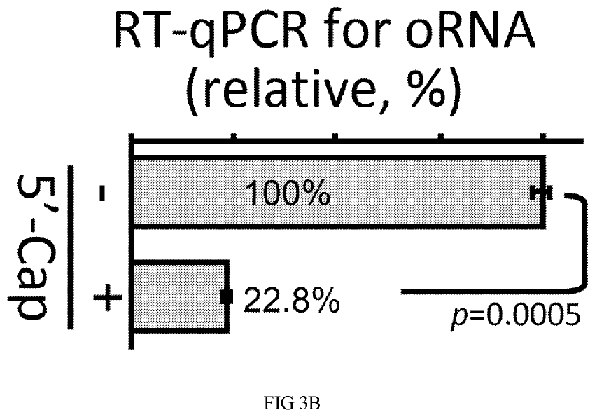

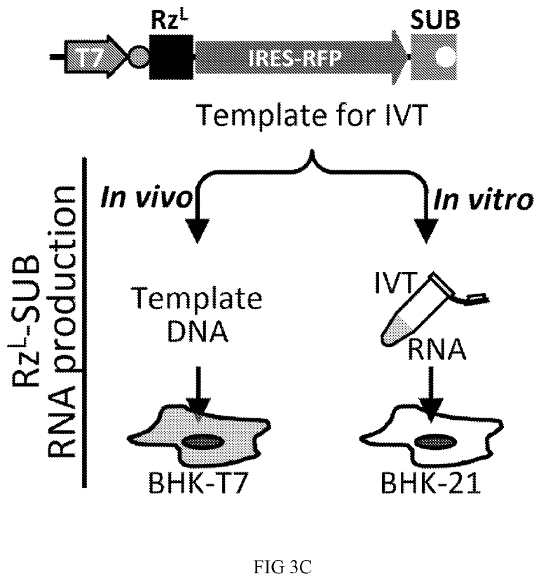

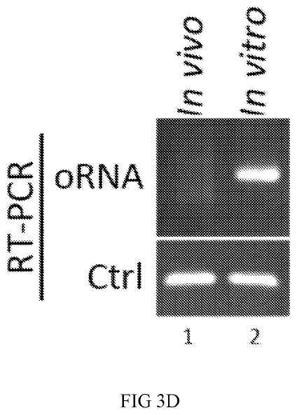

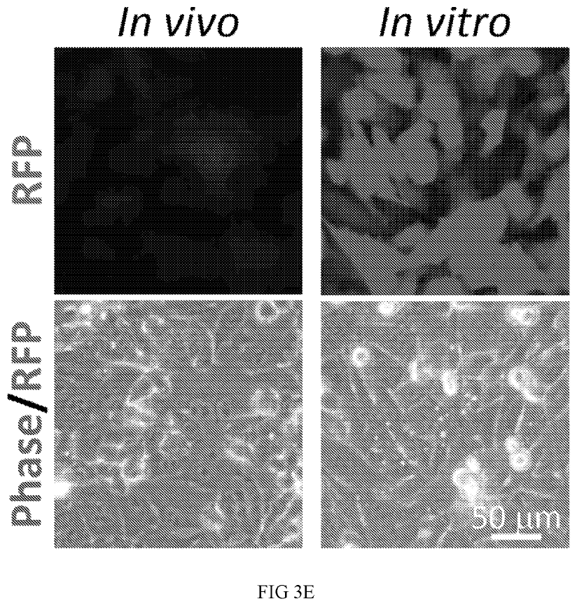

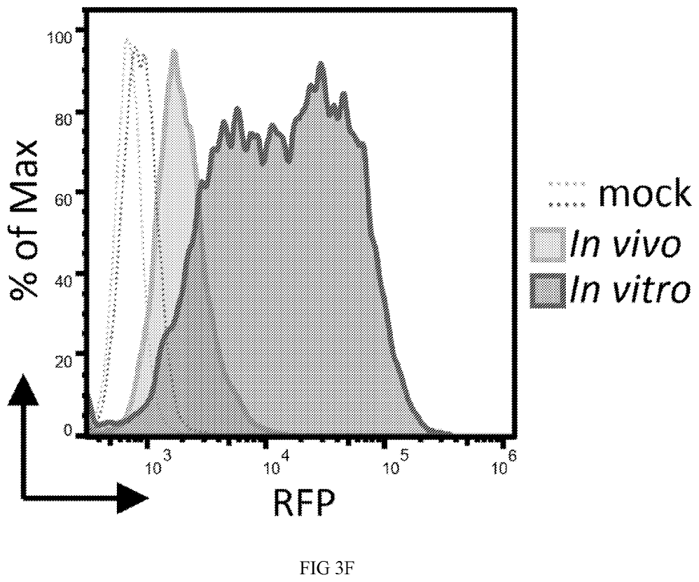

N7-methyl-guanosine-5′-ppp-5′-guanosine (m7G cap) was applied to mask the 5′-ppp and remove its circularization ability (FIG. 3A, B). Consistent with this, the design of Rz^{L}-SUB failed to form oRNA by T7 RNA polymerase in vivo because the transcribed RNA was 5′-Capped immediately in the transfected cell (FIG. 3C, D). Consequently, the RNA in circular form has more translation capability than linear in the cell (FIG. 3E, F) despite bearing IRES in both formats. Thus, the Rz^{L}-SUB method is capable of circularizing an RNA long enough in length for protein expression.

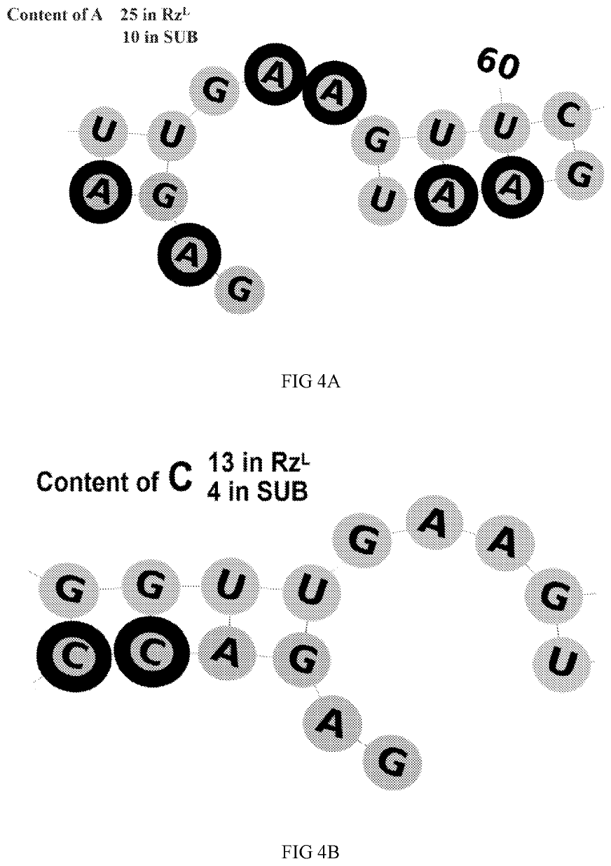

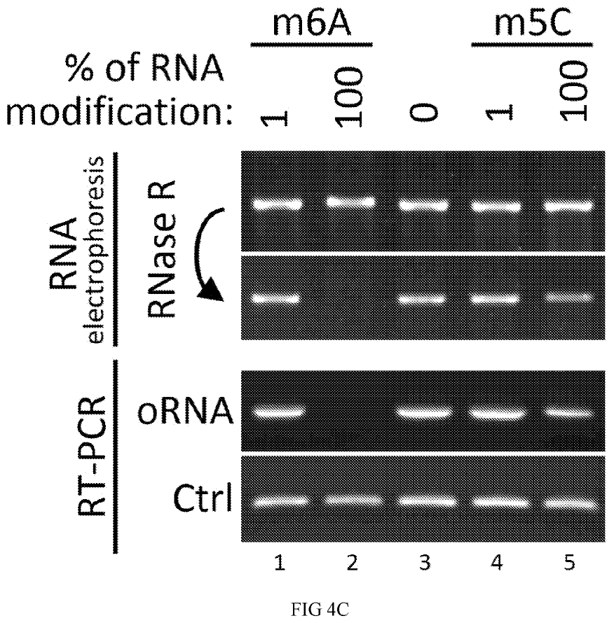

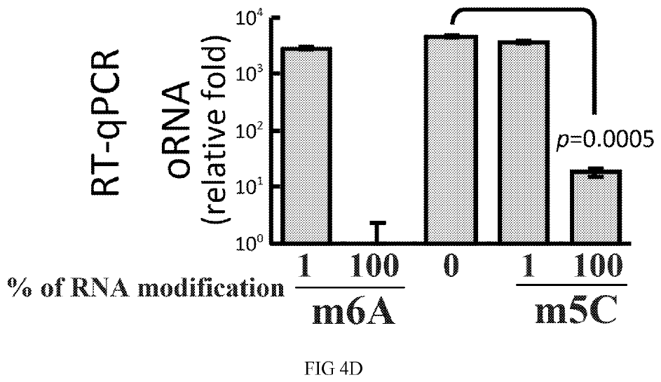

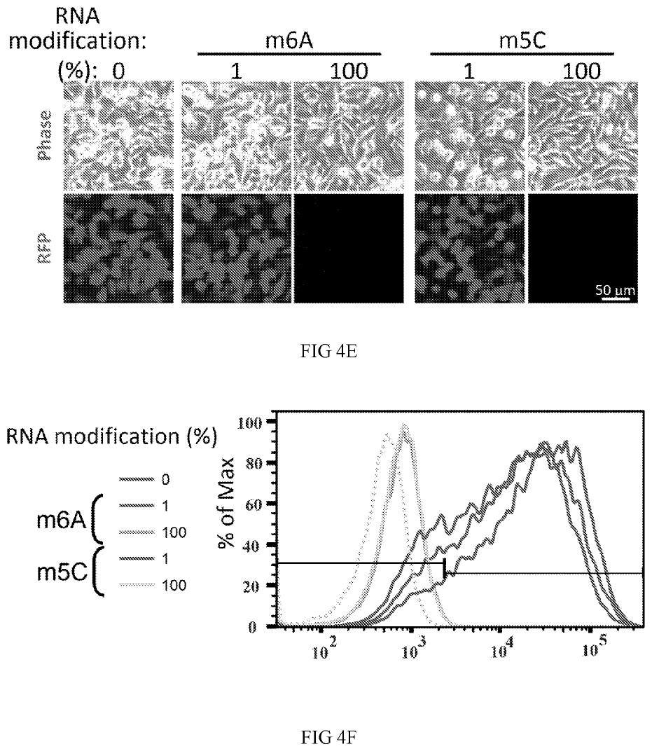

Since modifications in the Rz^{L} and SUB regions could likely affect the circularization and the contents of A and C were not equivalent within the region (FIG. 4A, B). Two common eukaryotic RNA modifications, N6-methyladenosine

(m6A) and 5-methylcytosine (m5C) were enrolled to verify whether modified RNA is tolerated in the Rz^{L}-mediated oRNA formation. As low as 1% A or C modification barely affecting the oRNA formation, which was perfectly blocked by replacing all the A with m6A (FIG. 4C, D). Thus, the RNA with 100% m6A modification remained linear and was inert in protein expression (FIG. 4E, F). In the case of replacing all the C with m5C, the oRNA formation was partially prohibited (FIG. 4C, D, lane 5). However, the m5C-modified oRNA remained incapable of protein expression (FIG. 4E, F), probably because of the m5C-mediated translation inhibition.

### Example 4. Three Strategies for Bicistronic Expression of Fluc and GFP

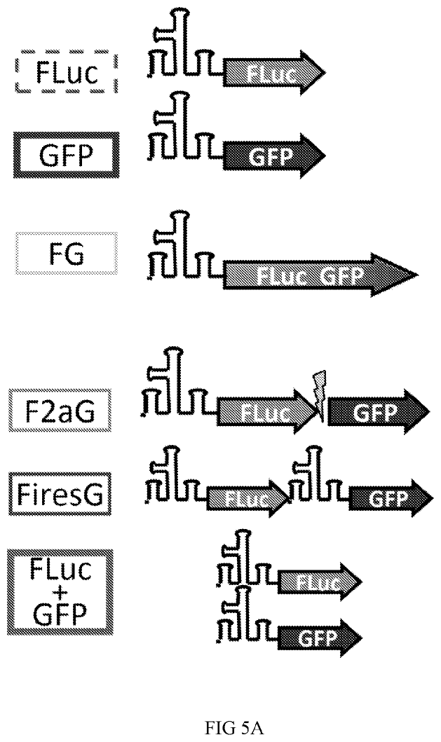

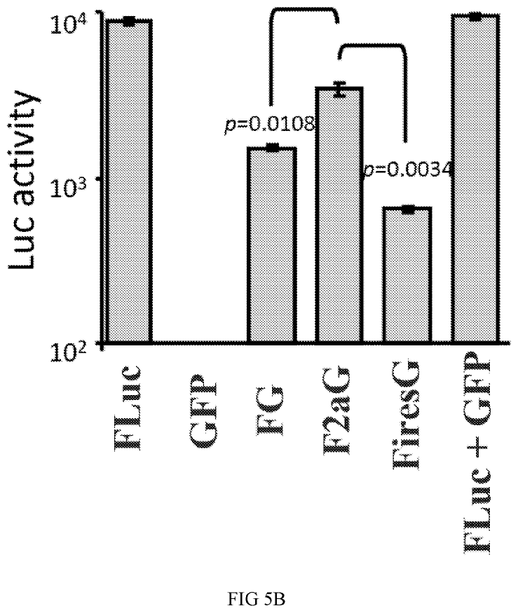

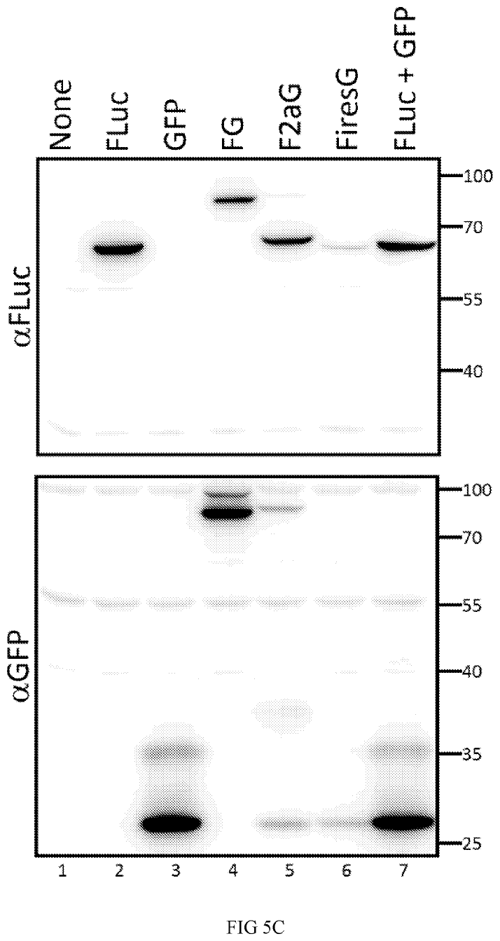

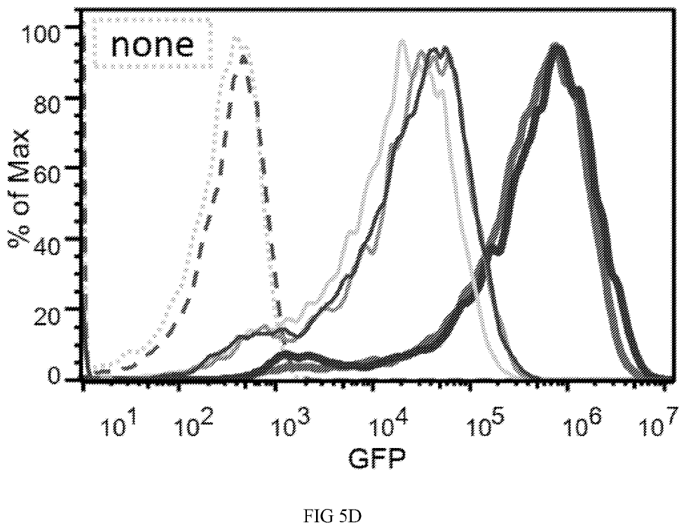

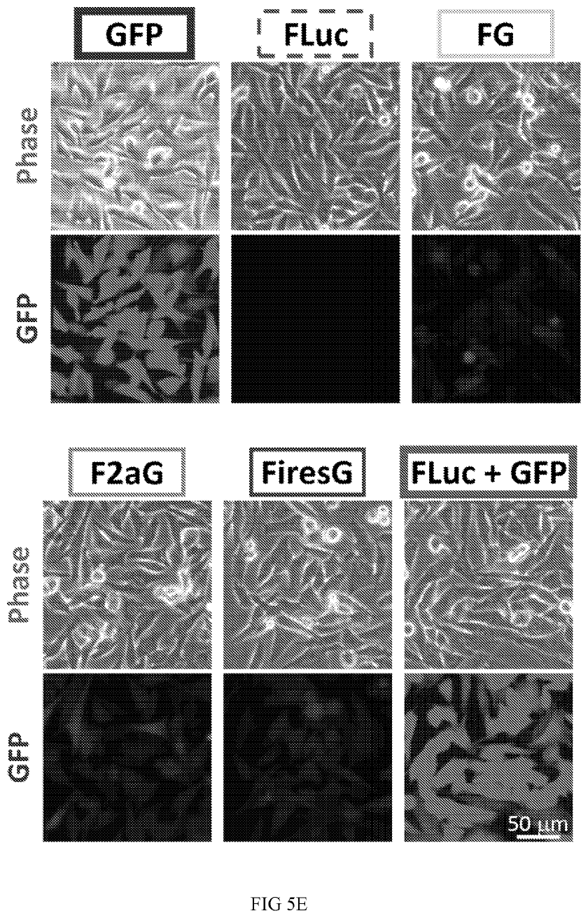

In this and following examples, three strategies were enrolled for the bicistronic expression of FLuc and GFP: fuse the two in a single protein (FG), separate the two by 2a-mediated ribosome skipping (F2aG), or introduce another IRES between the two (FiresG) (FIG. 5A). FLuc activity was attenuated by fusion with GFP (FIG. 5B, FLuc vs. FG) that could be slightly rescued by separating the FLuc from GFP with a 2a peptide (FIG. 5B, C). Unexpectedly, the dual-IRES strategy rendered FLuc the lowest level of protein expression among the three strategies (FIG. 5B, C). Consistently, flow cytometry (FIG. 5D) and fluorescence microscopy (FIG. 5E) analysis of the GFP expression showed that it was not because of variations in transfection efficiency but because of different expression capabilities. To overcome this, two separated oRNA were co-transfected in which either FLuc or GFP was driven by a single IRES (FLuc+GFP) and found they worked well for each other in the transfected cells (FIG. 5). Thus, the IRES-driven oRNA translation could be interfered with by another identical IRES in cis, not trans.

### Example 5: Viral RNA Comparable Translation could Bestow oRNA Antiviral Potentials

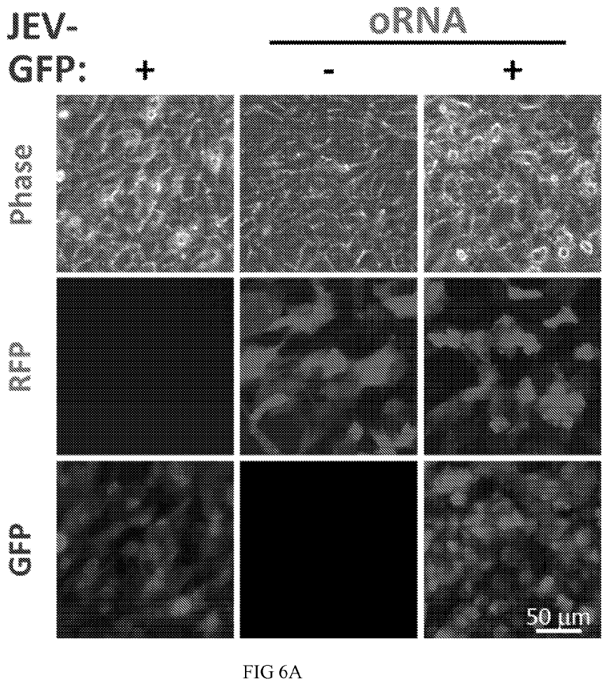

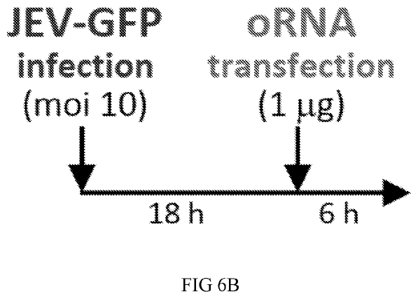

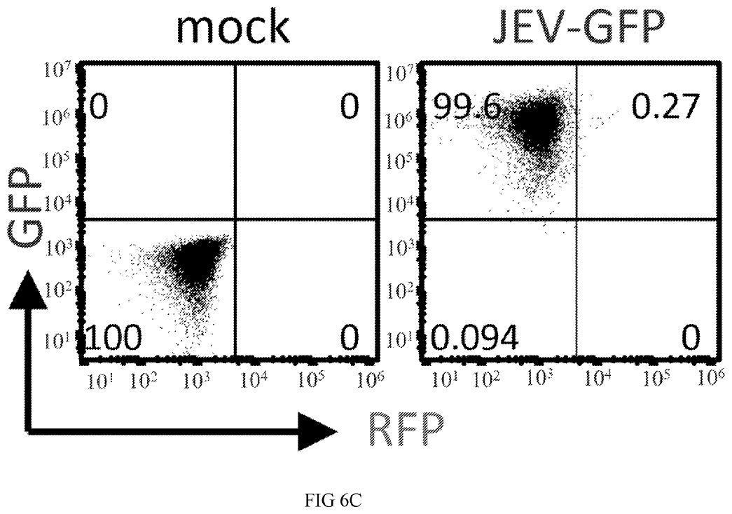

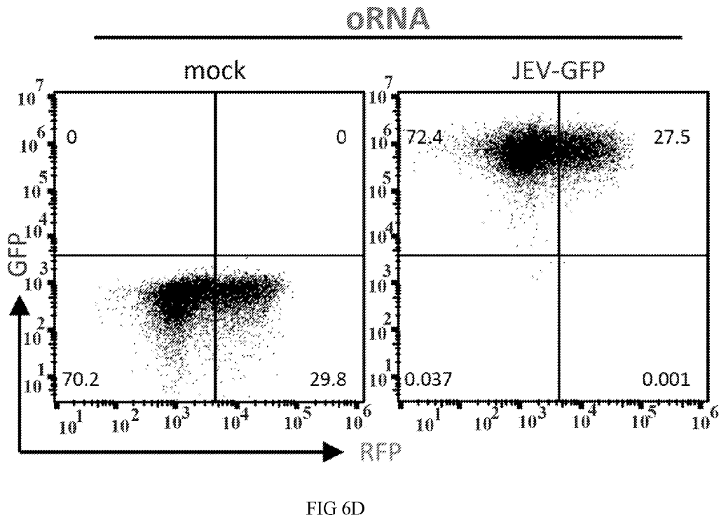

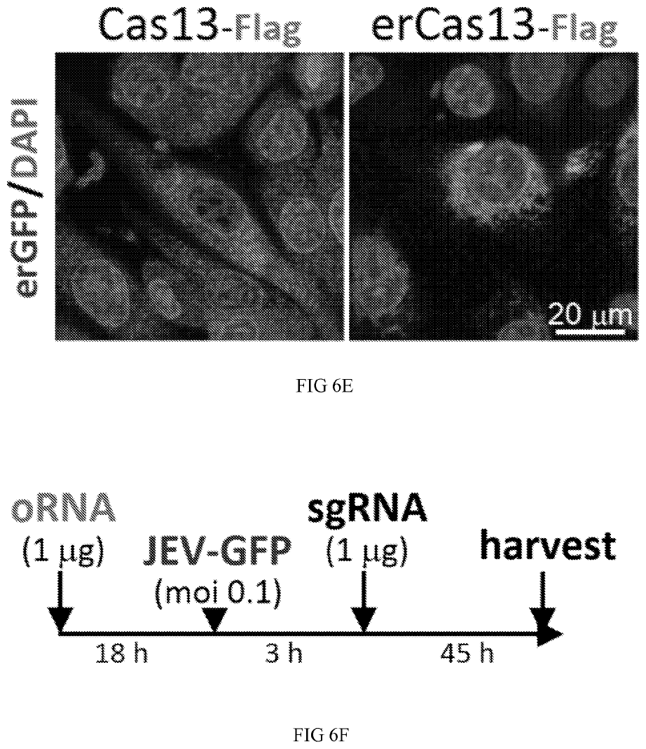

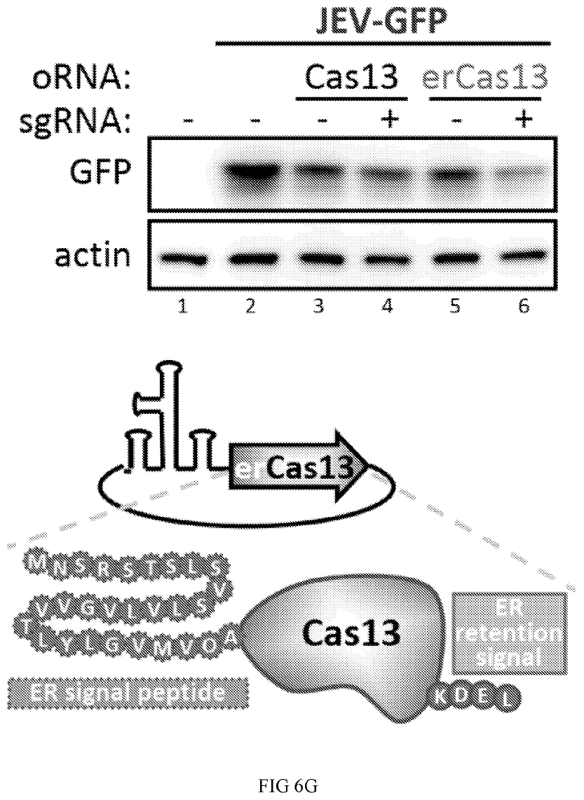

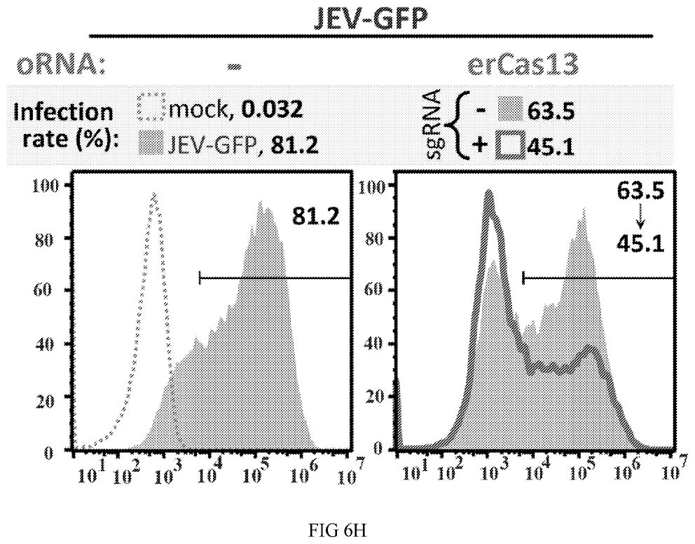

A recombinant JEV harboring GFP (JEV-GFP) whose 5′-capped RNA genome undergoes continuous replication and efficient translation. The GFP was readily detected at 24 h of the infection with or without oRNA transfection (FIG. 6A, B). Flow cytometry consistently showed marginal effects of JEV-GFP infection on oRNA translation in the experimental model of infection-transfection and vice versa (FIG. 6C, D). Thus, IRES-driven oRNA translation is comparable to a cap-dependent self-replicating RNA in trans, which is the prerequisite against the virus successfully via expressing antiviral proteins.

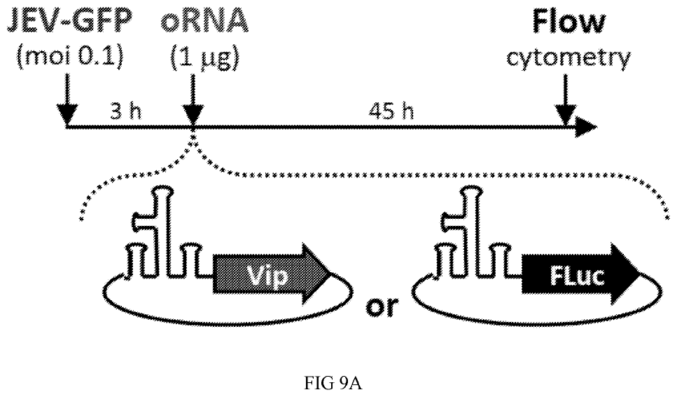

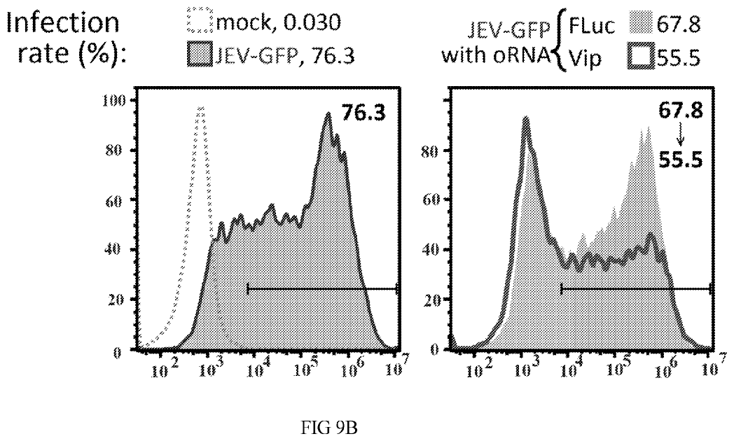

Next, an oRNA overexpressing the known broad-spectrum antiviral protein viperin for virus inhibition (FIG. 9A). The GFP positive rate reached approximately 76% at 48 h after JEV-GFP infection with low moi (FIG. 9B left panel). Introducing control oRNA (FLuc) reduced a certain level of GFP, which was further attenuated by introducing the oRNA encoding viperin (FIG. 9B right panel). Thus, the delivery of a natural antiviral protein by oRNA could effectively inhibit the virus.

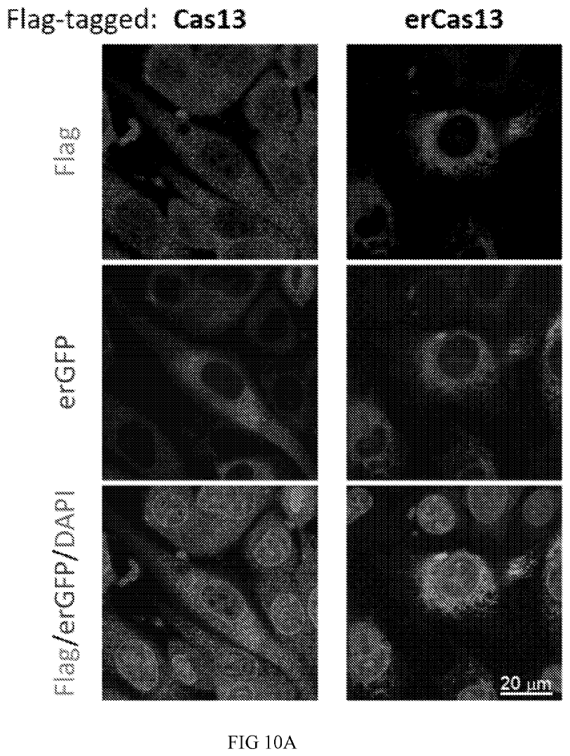

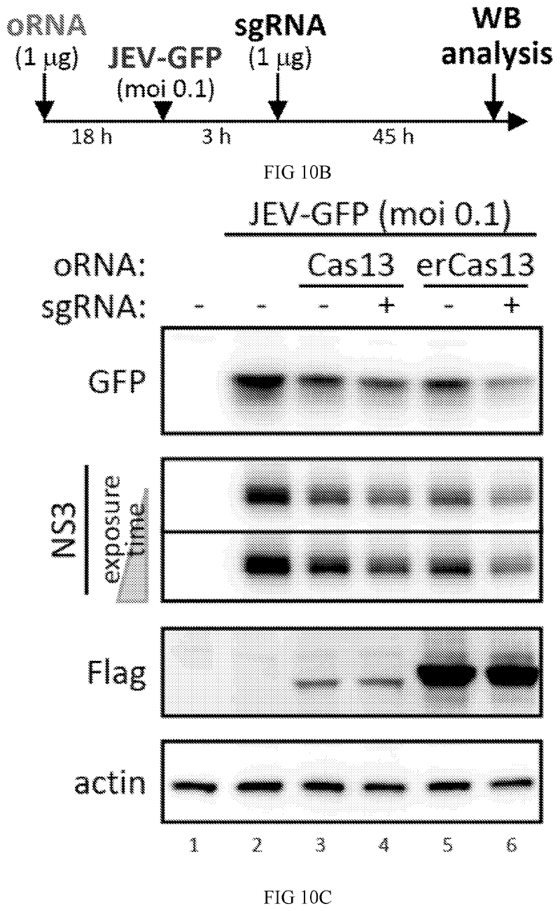

To have a specific-targeting antiviral, oRNA was used to express the RNA-guided RNA nucleases Cas13 (FIG. 6E and FIG. 10A left panel) first and then introduced the GFP-targeting single guide RNA (sgRNA) (FIG. 6F, upper panel). While GFP and JEV NS3 were readily detected after JEV-GFP infection, a certain level of antiviral activity resulting from Cas13 oRNA delivery was noticed (FIG. 6G and FIG. 10C lane 2 vs. 3). Although Cas13 oRNA with sgRNA lowered the NS3 level more than the control RNA (FIG. 10C lane 3 vs. 4), the difference was marginal by blotting GFP (FIG. 6G, lane 3 vs. 4). Regarding the orthoflavivirus RNA that may replicate and be concealed in the ER-associated membrane structures, the Cas13 was further modified to target ER (erCas13) (FIG. 6G, lower panel). When the infection rate reached ~81% in the group without any RNA delivery (FIG. 6H, left panel), delivery of the erCas13 oRNA alone showed a basal antiviral activity suppressing the virus infection rate to ~63% (FIG. 6H, right panel). Applied the sgRNA under such circumstances, erCas13 could further suppress the infection rate toward as low as ~45%, which worked better than the parental Cas13 in the cytosol for a functional antiviral activity against the JEV-GFP virus (FIG. 6G and FIG. 10C).

From the above description, one skilled in the art can easily ascertain the essential characteristics of the present invention and, without departing from the spirit and scope thereof, can make various changes and modifications of the invention to adapt it to various usages and conditions. Thus, other embodiments are also within the scope of the following claims.

1. A plasmid for producing a circular RNA having an arrangement of the region, wherein said arrangement is a 5′ polymerase promoter sequence, a nucleic acid sequence encoding a ligase ribozyme RNA, a nucleic acid sequence encoding an internal ribosome entry site (IRES), a nucleic acid sequence encoding an effector molecule, and a nucleic acid harboring a free OH group at the very 3′ end for conjunction to the ligase ribozyme RNA aforementioned.

2. The plasmid of claim 1, wherein the 5′ polymerase promoter sequence is prokaryotic T7 promoter, T3 promoter, SP6 promoter, Lac promoter or eukaryotic RNA polymerase I promoter.

3. The plasmid of claim 1, wherein the ligase ribozyme RNA is an cis-acting ligase ribozyme.

4. The plasmid of claim 1, wherein the IRES sequence is selected from a virus, a cell, an artificial sequence or a gene selected from the group consisting of CVB3 virus, EV71 virus, EMCV virus, PV virus, and CSFV virus.

5. The plasmid of claim 1, wherein the arrangement further comprises a substrate sequence of the ligase ribozyme RNA.

6. The plasmid of claim 1, wherein the substrate sequence of the ligase ribozyme RNA is SUB sequence, SUB_2 sequence, or SUB_3 sequence.

7. The plasmid of claim 1, wherein the effector molecule sequence is microRNA sequence, microRNA sponge sequence, receptor protein sequence, target protein sequence, antigen sequence, protein enzyme sequence, functional protein sequence, or bicistronic protein expression sequence.

8. A method for producing a circular RNA, wherein said method comprising the steps of:
(a) providing the plasmid of claim 1;
(b) transcribing an RNA base on the plasmid and
(c) allowing the RNA to self-ligation, thereby producing the circular RNA.

9. The method of claim 8, wherein the 5′ polymerase promoter sequence is prokaryotic T7 promoter, T3 promoter, SP6 promoter, CMV promoter, or eukaryotic RNA polymerase I promoter.

10. The method of claim 8, wherein the ligase ribozyme RNA is an cis-acting ligase ribozyme.

11. The method of claim 8, wherein the IRES sequence is selected from a virus, a cell, an artificial sequence or a gene selected from the group consisting of CVB3 virus, EV71 virus, EMCV virus, PV virus, and CSFV virus.

12. The method of claim 8, wherein the arrangement further comprises a substrate sequence of the ligase ribozyme RNA.

13. The method of claim 12, wherein the substrate sequence of the ligase ribozyme RNA is SUB sequence, SUB_2 sequence, or SUB_3 sequence.

14. The method of claim 8, wherein the effector molecule sequence is microRNA sequence, microRNA sponge sequence, receptor protein sequence, target protein sequence, antigen sequence, protein enzyme sequence, functional protein sequence, or bicistronic protein expression sequence.

15. The method of claim 8, wherein the plasmid is transcribed in vitro or in vivo.

16. The method of claim 8, wherein the RNA further comprising a conjunction ring.

17. The method of claim 16, wherein the conjunction ring consists of three nucleotides for bridging at the middle and two overhang nucleotides at the 5′ end.

18. The method of claim 17, wherein the RNA further comprising a 5′-end-protruding overhang ends.

19. A circular RNA molecule generated according to claim 8.
